(19) United States
(12) Patent Application Publication
(10) Pub. No.: US 2006/0034890 A1
(43) Pub. Date: Feb. 16, 2006

(54) Device for ocular delivery of active principles by the transpalpebral route

(76) Inventors: Pierre Roy, Paris (FR); Alain Kleinsinger, Neuilly - Sur- Seine (FR)

Correspondence Address: BLAKELY SOKOLOFF TAYLOR & ZAFMAN/PDC, 12400 WILSHIRE BOULEVARD, SEVENTH FLOOR, LOS ANGELES, CA, 90025, US

(21) Appl. No.: 10/538,678

(22) PCT Filed: Dec. 11, 2003
(86) PCT No.: PCT/IB03/06357

§ 371 Date: Jun. 10, 2005

(30) Foreign Application Priority Data
Dec. 11, 2002 (FR) 02/15646

(51) Int. Cl. A61F 2/00

(52) U.S. Cl. 424/427

(57) ABSTRACT

The invention relates to a device for ocular delivery of active principle(s) (1) comprising a first outer layer (2) which is essentially non-leaking, and a second inner layer (3) which has a surface (7) able to come into contact with at least one eyelid. characterized in that the second layer comprises at least one active principle intended to be delivered to the patient through the eyelid.

The invention relates to a device for ocular delivery of medicaments, in particular by the transpalpebral route.

In recent years, important inventions have been made in the diagnosis and treatment, mainly by surgery, of eye diseases, for example diabetic retinopathy, glaucoma, macular degeneration, and detachment of the retina. As physiological knowledge of these diseases progresses, numerous active principles have proven of interest and are in the course of development, making it possible to reduce treatment by surgery.

In addition to the numerous antibiotics, antivirals, and other antifungals presently used to treat infections of the retina and of the vitreous body, many anti-inflammatory and anti-cancer agents appear promising in the treatment of proliferative diseases.

Similarly, the advances made in local anaesthesia and in surgical techniques have reduced the use of general anaesthesia and its associated risks. Nevertheless, there remains a compromise between the side effects, the duration of anaesthesia, the quality of akinesia and the pain experienced by the patient.

However, the delivery of medicaments into the eye remains problematic.

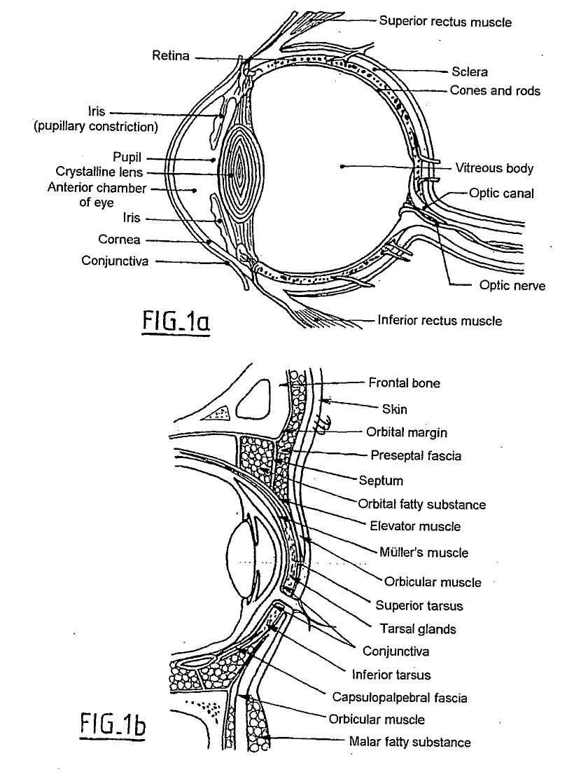

This is because the eye (of which an anatomical cross section is illustrated in FIG. 1) is made up of a number of defensive barriers which greatly limit penetration into the intraocular tissues:
the lachrymal film is the first line of defence encountered by medicaments applied topically: it dilutes and drains the medicament,
the cornea made of an epithelium of lipophilic character which serves as a barrier to the hydrophilic substances, and of the hydrophilic stroma which serves as a barrier to the lipophilic substances and as a reservoir for the hydrophilic substances,
the conjunctiva and the sclera, highly vascularized tissues which encourage a systemic passage of the active principles,
the crystalline lens containing the aqueous humor rich in proteins capable of binding to the active principles,
the iris which serves as a reservoir for the lipophilic substances and which very slowly releases these,
the retina protected by the blood-retina barrier.

The techniques used for administering active principles into the eye may presently be described as follows:
The systemic route (oral or intravenous), sometimes by bolus (high dose, short duration), does not give a high concentration in the eye (less than 1% of active principles) because the blood-retina barrier is relatively impermeable to numerous active principles. Moreover, these drugs may have considerable side effects on other organs of the human body.
Direct injections around the eye (peribulbar or retrobulbar) have a relatively low efficiency because little active principles actually passes into the eye, and they are not without side effects such as haemorrhages, even accidental perforation of the eyeball.
Intraocular injections (directly into the eye) cause trauma and the drug is rapidly diluted and disappears from the vitreous body within a few days. Moreover, this mode of administration presents certain risks such as infection, cataract, and detachment of the retina. Conditions such as glaucoma cannot be treated in this way because of the risk of increased intraocular pressure.
The intraocular implant for controlled release of medicaments implanted in the vitreous body partially solves the aforementioned problem but nevertheless has disadvantages, on the one hand of moving freely in the vitreous body and thus risking touching the retina, with consequent increase in the local concentration of active principles to a toxic level, and, on the other hand, of having to be replaced regularly. Moreover, the implant delivers a constant amount of medicament, and this amount cannot be modulated as a function of the course of the pathology. A bioerodable or biodegradable implant does not have to be replaced. It is possible to suture the implant, but this requires a relatively wide incision of about 5 mm and poses risks of endophthalmitis or detachment of the retina.
Topical application by drops does not treat the posterior segment of the eye, because the penetration of the active principle is very limited and does not permit therapeutic concentrations beyond the anterior segment of the eye. Moreover, as the tears rapidly wash the active principle, the applications have to be repeated frequently.
Photodynamic therapy is a technique which consists in injecting an active principle systemically and activating it locally using a laser of a certain wavelength. Among its disadvantages, the patient has to remain in complete darkness because of his/her general hypersensitivity to light, the active principle has to be modified by addition of a photosensitive agent which blocks its activity until activation by the laser, and the physician must have relatively expensive equipment to hand.

In another field, the techniques of local anaesthesia, apart from general anaesthesia of non-cooperative patients for long or short durations, are four in number, the last one being in the course of development. These techniques may sometimes be combined with one another:
Retrobulbar anaesthesia consisting in injecting the anaesthetic with the aid of a needle to the posterior of the eyeball, inside the space formed by the oculomotor muscles. This technique may lead to complications including perforation of the eyeball, retrobulbar haemorrhage, damage to the optic nerve, accidental intravascular injections (leading to the risk of poor anaesthesia or cardiac or respiratory arrest, depending on the mixture used), or vascular occlusions of the retina. However, the quality and duration of the anaesthesia are good.
Peribulbar anaesthesia, consisting in injecting the anaesthetic with the aid of a needle around the eyeball outside the space formed by the oculomotor muscles. This technique leads to the same complications as the preceding one, but less commonly, since the penetration of the needle is less deep. The results of this type of anaesthesia are as good as those of the preceding type.
Topical anaesthesia consists in instilling the anaesthetic into the conjunctival fornices. This technique does not lead to the preceding complications but, compared to the above methods, provides anaesthesia of shorter duration (admittedly sufficient for many operations), of lesser quality (there is more eye mobility after application of the anaesthetic), and also causes the patient perioperative and postoperative pain. It is commonly necessary to use sedatives which are administered intravenously and may cause complications (respiratory arrest, for example). In this case, the presence of an anaesthetist is strongly recommended, which is the same as the preceding case.
Retrobulbar anaesthesia by catheter consists in placing a catheter of the peridural type (28 Ga, or 0.4 mm diameter to 1.0 mm) in the retrobulbar space or peribulbar space via a needle in order to be able to inject the anaesthetic for operations of long duration (more than 60 minutes) or to administer it continuously, even in the postoperative phase. The risks of error remain a priori identical to those of the techniques of retrobulbar and peribulbar anaesthesia above.

The object of the invention is to make available a device for ocular delivery of active principles which is easy to use, making it possible to obtain a concentration of active principles which is sufficient for certain treatments in the intraocular or periocular tissues, while at the same time solving or avoiding the aforementioned problems.

To this end, the invention proposes a device for ocular delivery of active principles comprising a first, outer layer which is essentially non-leaking, and a second, inner layer which has a surface able to come into contact with at least one eyelid, characterized in that the second layer comprises at least one active principle intended to be delivered through the eyelid.

It is advantageous, though optional, for the device for ocular delivery to have at least one of the following characteristics:
the first, non-leaking layer has an inner face, and the second layer partially covers the inner face,
a part of the inner face not covered by the second layer can be covered with a cutaneous adhesive,
the device for ocular delivery comprises at least two parts,
the device has an orifice at its centre,
it additionally comprises a reservoir able to be brought into communication with the second layer via means of communication,
the means of communication is a protective seal able to insulate the reservoir of the second layer in a leaktight manner before its withdrawal,
the layer is a rigid or semi-rigid shell.
the rigid or semi-rigid shell has, on its outer face, application means.

Other characteristics and advantages of the invention will appear on reading the following description of a preferred embodiment and variants thereof. In the attached drawings:

FIG. 1a is an anatomical cross section of an eye,

FIG. 1b is an anatomical cross section of the eyelids,

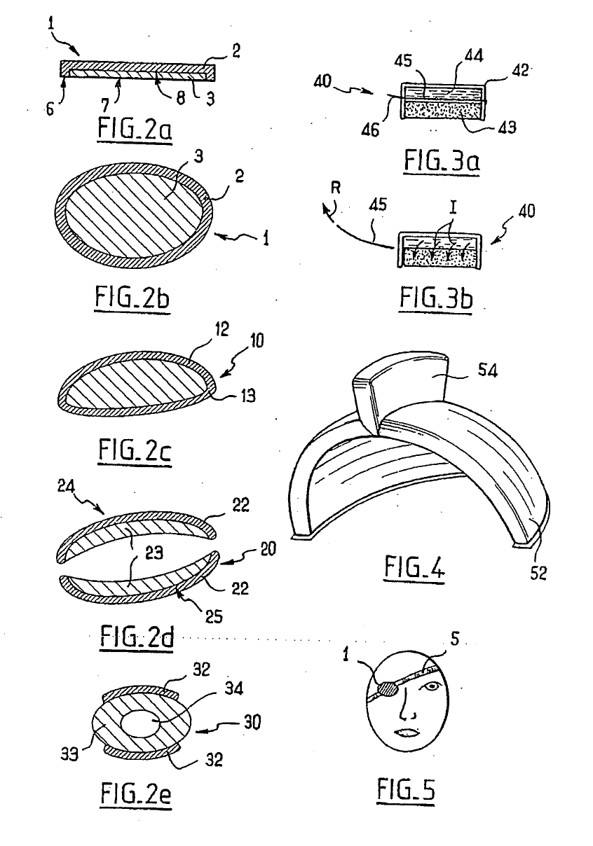

FIG. 2a is a cross section through a first embodiment of the invention,

FIG. 2b is a plan view of the first embodiment in FIG. 2a,

FIGS. 2c to 2e are plan views of variants of the embodiment in FIG. 2b,

FIGS. 3a and 3b are cross sections through a second embodiment of the invention,

FIG. 4 is a view, in three dimensions, of an alternative embodiment of the outer part of the invention,

FIG. 5 is a diagrammatic view showing the use of the device according to the invention, and

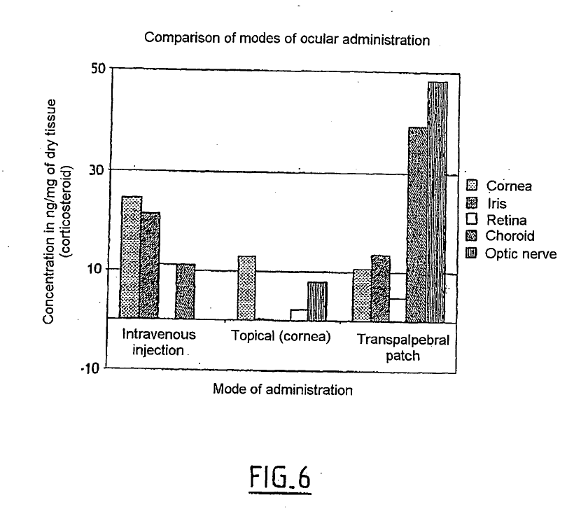

FIG. 6 is a graph comparing the results of different modes of administration of active principles into the eye, according to the part of the region of the eye considered.

Generally, the shape of a device for delivery of active principles into the eye is configured in such a way as to cover the eye, with the eyelids closed, that is to say the rough equivalent of the surface area of an oval disc measuring about 35 mm by 40 mm.

Referring to FIGS. 2a and 2b, we will describe a first preferred embodiment of a device 1 for ocular delivery of active principles according to the invention. The device 1 comprises two layers 2 and 3 of materials:
a first layer 2 of material which is essentially non-leaking while being flexible so as to conform optimally to the anatomy of the eye for which the delivery device according to the invention is intended to be used. This can be a film of polymer material such as polyethylene, polypropylene, polyurethane, polyvinyl chloride, etc. In one variant, the first layer 2 can be rigid and preformed anatomically. In this case, the first layer 2 forms a rigid or semi-rigid shell which can be produced by injection in a mould or by thermoforming.
A second layer 3 forming a reservoir of active principles intended to be delivered in the ocular region. This second layer comprises a face 7 able to be in direct contact with the eyelid or eyelids covering the eyeball. The second layer 3 can be made of a preferably absorbent material, for example a foam of polymer material having a hydrophilic character (polyurethane, cellulose acetate), a foam of natural material having a fibrous character (paper, cotton, etc.), or a hydrogel. The property common to all of the aforementioned materials is a high absorption capacity combined with great flexibility so as to conform intimately to the shape of the eyelid or eyelids with which the second layer is able to come into contact.

The first layer 2 has an inner face 8 which can be partially or completely covered by the second layer 3. In the case where only part of the inner face 8 of the first layer 2 is covered by the second layer 3, the complementary, uncovered part 6 of the inner face 8 can receive a cutaneous adhesive with which the device for ocular delivery of active principles 1 is held on the eyelid or eyelids.

Referring to FIG. 2b, the device for ocular delivery of active principles 1 is of a general oval shape so as to cover both eyelids, once these have been closed across the eyeball. Moreover, the second layer 3 only partially covers the inner face 8 of the first layer 2, so as to leave a ring which surrounds this second layer 3 and on which the cutaneous adhesive is placed, as described above.

Referring to FIG. 2c, a variant embodiment 10 of the device for delivery of active principles has a structure similar to the structure of the ocular delivery device 1 described above, but here the device 10 has a shape which covers only the upper eyelid once the latter has been closed across the eyeball. The second layer 13 partially covers the first layer 12 so as to leave a ring able to receive a cutaneous adhesive surrounding the second layer 13.

Referring to FIG. 2d, this illustrates another variant embodiment 20 of the device for ocular delivery of active principles according to the invention. In this alternative embodiment, the delivery device 20 is composed of two parts 24 and 25 which may be identical. Each part 24 and 25 has a first layer 22 and a second, absorbent layer 23 which are arranged in such a way that the layer 23 partially covers the layer 22 so as to leave, in the upper periphery of part 24 and in the lower periphery of part 25 of the layer 22, a strip which is able to receive a cutaneous adhesive for placing the ocular delivery device 20 on the eyelids.

Referring to FIG. 2e, this illustrates another variant embodiment 30 of the delivery device according to the invention. This device 30 has a general oval shape roughly similar in its dimensions to those of the ocular delivery device 1 described above. Again, the second layer 33 partially covers the first layer 32 which extends beyond the layer 33 in the form of two strips, respectively in the upper part and in the lower part, which strips are able to receive a cutaneous adhesive for putting the device in place. Moreover, the device 30 has an orifice 34 situated at about the centre of the device. Once the patch has been fitted, this orifice 34 means that there is no absorbent layer 33 opposite the cornea of the eyeball, so that it is possible to avoid delivering active principles directly to this specific site of the ocular region.

All of the embodiment variants illustrated in FIGS. 2a to 2e are designed to be flexible. Before being placed on the eyelids, these devices have a plane shape and, because of their flexibility, they are able to optimally conform to the shape of the eyelids closed across the eyeball during fitting.

Referring to FIGS. 3a and 3b, we will now describe a second embodiment of a device for ocular delivery of active principles according to the invention. It should be noted that the general shape of this embodiment is similar to the general shape of the preceding embodiment. As before, the device for ocular delivery of active principles 40 has a first, non-leaking layer 42 and a second layer made of absorbent material 43. Moreover, the ocular delivery device 40 has a reservoir 44 situated essentially between the first, non-leaking layer 42 and the absorbent material 43. In addition, before use, the reservoir 44 is kept isolated from the absorbent material 43 in an essentially non-leaking manner by a protective seal 45 which extends beyond the non-leaking layer 42 in the form of a tongue 46 serving as a means of using the protective seal 45. In use, the ocular delivery device 40 is placed on the eyelid so that a face 47 of the second, absorbent layer 43 comes into contact with the eyelid or eyelids closed across the eyeball of the patient to be treated. Once the ocular delivery device 40 has been put in place in this way, the protective seal 45 is removed by pulling the tongue 46 in the direction of the arrow R, the absorbent layer 43 thus coming into contact with the contents of the reservoir 44. The contents of the reservoir 44 then soak the absorbent material 43 in the direction indicated by the arrows I. This initiates the ocular delivery of the active principle which has previously been contained in the reservoir 44 and which has soaked the absorbent material 43.

In a variant of one of the embodiments described above, the first, non-leaking layer 2 can be rigid. An illustrative embodiment of a rigid, non-leaking layer of this kind is illustrated in FIG. 4. The non-leaking layer in this case is in the form of a rigid or semi-rigid non-leaking shell 52 equipped on an outer face with grip means 54, which grip means permit and facilitate the use of the device for ocular delivery, thus equipped, on the patient to be treated.

It is advantageous, though optional, to use such a rigid or semi-rigid shell together with the second embodiment 40 of the device for ocular delivery of active principles described above. This is because a rigid or semi-rigid shell of this kind can clearly define the reservoir 44 which, before use, will contain the active principle in liquid form. It should be noted that, in this case, numerous systems for bringing the reservoir 44 into communication with the material 43 at the time of use can be employed and are known to the person skilled in the art.

Referring to FIG. 5, in the case of relatively prolonged use, that is to say when the ocular delivery device according to the invention is to be placed on the eyelids for a relatively long time, the device for ocular delivery of active principles 1 can be equipped with an elastic strap 5, for example, in order to secure it on the patient for the time necessary. In variant embodiments, this elastic strap 5 can be replaced by the arms of spectacles, headbands, etc. In addition, this assembly can be arranged so as to be able to treat both eyes of a patient simultaneously.

Referring to FIG. 6, we will now examine the efficacy of the device for ocular delivery of active principles according to the invention, as described above, compared with two other types of administration, namely intravenous injection and topical administration, such as have been described in the preamble of this description.

For each of the modes of administration considered, we measured the concentration of active principles in the cornea, in the iris, in the retina, in the choroid and at the optic nerve. The measurements were carried out about 10 minutes after administration of an active principle (a corticosteroid) into the region of the eyeball of a rabbit.

As regards the mode of administration by intravenous injection, it will be noted that the concentration of active principles is fairly moderate in the cornea and the iris, low in the retina and choroid, and zero at the optic nerve.

With topical administration on the cornea, the concentration of active principles is low in the cornea, zero in the iris and the retina, and very low in the choroid and the optic nerve.

Finally, the mode of administration using a device for ocular delivery of active principles according to the invention gives results which are low in the cornea and iris and very low in the retina, but provides excellent results in the choroid and the optic nerve.

It should be noted that, for the third mode of administration, the tests were carried out on rabbits and involved using a device for ocular delivery of active principles according to the invention of oval shape and measuring in the order of about 15 mm by 20 mm, applied to the eyelid for 10 minutes before measuring the concentrations of active principles. The ocular delivery device had a structure similar to that illustrated in FIG. 2e, hence the low concentrations found in the cornea and iris essentially.

Moreover, it has been found that by targeting certain areas of the eyelid, the active principle tended to penetrate more into the periocular space, where the oculomotor muscles are situated, toward the posterior part of the orbit, where the optic nerve is situated, circumventing the eyeball. Thus, by acting on the general shape of the device for ocular delivery of active principles according to the invention, it is possible to promote penetration into certain tissues for a given objective, such as anaesthesia of the muscles or the neuroprotectors of the optic nerve, for example.

Of course, many modifications may be made to the invention without thereby departing from the scope thereof.

1. Device for ocular delivery of active principle(s) (1; 10; 20; 30; 40) comprising a first outer layer (2; 12; 22; 32; 42) which is essentially non-leaking, and a second inner layer (3; 13; 23; 33; 43) which has a surface (7) able to come into contact with at least one eyelid, characterized in that the second layer comprises at least one active principle intended to be delivered through the eyelid.

2. Device according to claim 1, characterized in that the first, non-leaking layer has an inner face (8), and the second layer (3) partially overlies the inner face (8).

3. Device according to claim 2, characterized in that a part (6) of the inner face (8) not overlied by the second layer (3) can be overlied with a cutaneous adhesive.

4. Device according to one of claims 1 to 3, characterized in that the device for ocular delivery (20) comprises at least two parts (24, 25).

5. Device according to one of claims 1 to 4, characterized in that the device has an orifice (34) at its centre.

6. Device according to one of claims 1 to 5, characterized in that it additionally comprises a reservoir (44) able to be brought into communication with the second layer (43) via means of communication (45, 46).

7. Device according to claim 6, characterized in that the means of communication is a protective seal (45) able to insulate the reservoir of the second layer in a non-leaking manner before its withdrawal.

8. Device according to one of claims 1 to 7, characterized in that the layer (2) is a rigid or semi-rigid shell (52).

9. Device according to claim 8, characterized in that the rigid or semi-rigid shell has, on an outer face, application means (54).
